(19) United States
(12) Patent Application Publication
(10) Pub. No.: US 2003/0195524 A1
(43) Pub. Date: Oct. 16, 2003

(54) Body tissue aspiration cannula

(76) Inventor: Gil Barner, Siler City, NC (US)

Correspondence Address: MORGAN LEWIS & BOCKIUS LLP, 1111 PENNSYLVANIA AVENUE NW, WASHINGTON, DC, 20004, US

(21) Appl. No.: 10/121,397

(22) Filed: Apr. 12, 2002

(51) Int. Cl. A61B 17/42

(52) U.S. Cl. 606/119; 604/264

(57) ABSTRACT

The invention relates to a body tissue aspiration cannula capable of being steam sterilizable comprising a tubular portion of a polymeric material with a flexural modulus of from about 100,000 psi to about 225,000 psi. It also relates to a novel cannula hub comprising a pair of opposing grip wings.

### BACKGROUND OF THE INVENTION

1. Field of the Invention

The present invention relates generally to a surgical aspiration cannula. In particular, the invention relates to a body tissue aspiration cannula having improved feel during use, may be chemical or steam sterilized, and which is more resistant to, bending, twisting, or kinking. The invention also relates to a novel hub design for an aspiration cannula which aids in insertion and removal of the vacuum source.

2. Description of Related Art

During the first trimester of pregnancy, vacuum aspiration is the standard surgical method available in the United States for induced abortion. In many underdeveloped countries, it is the only method available for safe induced abortion and the treatment of incomplete abortion and worldwide it is the most common method. Where available or preferred, vacuum is created for such aspiration via an electrical, vacuum pump. When an electrical vacuum pump is unavailable or not preferred, vacuum is usually created via a vacuum syringe. Once the source of vacuum is selected, the source of vacuum is connected to a body tissue aspiration cannula, which has one, or more body tissue aspiration apertures designed to aspirate the products of conception through the lumen of the tube, the tube having been inserted into the uterine cavity. A problem with using the body tissue aspiration cannula may come as it is inserted into the uterine cavity. It can be difficult to accomplish such insertion without use of a uterine dilator, especially if the cannula is too flexible. If the body tissue aspiration cannula is too flexible, the tendency is for the cannula to kink or deform at either one of the apertures or at the center of the cannula due to the pressure exerted on the cannula tip during use. For example, up to 10 pounds of pressure are applied to the tip of the tubular portion of the cannula during use. Pressure under 4 pounds is usually sufficient for kinking or deformation of such soft tubular portions. A body tissue aspiration cannula therefore needs to be sufficiently rigid so that it can be guided into place, yet at the same time, sufficiently flexible so as not to readily damage or penetrate body tissue. Accordingly, in an attempt to address these issues several different types of cannula are marketed and termed, generally, as having a tubular portion that is flexible, rigid or semi-rigid. Typically, these cannula are made of surgical grade polyethylene. Flexible cannula in the market today have a flexural modulus of less than about 80,000 psi while semi-rigid and rigid cannula have a flexural modulus greater than about 300,000 psi.

After the vacuum is engaged and the selected cannula positioned and repositioned to remove the products of conception through the apertures, the cannula may be disposed of or may be in certain circumstances, sterilized and reused. In order to withstand the strain of reuse, a body tissue aspiration cannula must not only have the desired flexibility or rigidity, it should also be able to withstand repeated sterilization by either steam or chemical sterilization methods. Further, to meet regulatory requirements, the material used to make the cannula must be surgical grade usually USP Class VI certified to insure being biocompatible with the tissue it comes in contact with.

It would be useful if there were a body tissue aspiration cannula that would have a stiffness between what are deemed a rigid and a flexible aspiration cannula to address the above concerns. It would be useful if this cannula had the ability to be repeatedly sterilized by chemical or steam regimens. It would also be useful if the body tissue aspiration cannula would resist kinking around the apertures during use, while still being relatively flexible enough to minimize any potential risk to surrounding tissue during use.

### SUMMARY OF THE INVENTION

The present invention addresses the needs and desires described above while providing improved performance parameters. In its broadest aspect, the invention relates to a body tissue aspiration cannula comprising a tubular portion having a proximal end, a distal end and a lumen extending therebetween, the distal end of the tubular portion being sealed. The tubular portion comprises a biocompatible, steam sterilizeable polymer having a flexural modulus of from about 100,000 psi to about 250,000 psi, with the tubular portion having one or more body tissue aspiration apertures disposed near the distal end. A hub portion comprising a biocompatible, steam sterilizable polymer is secured to the proximal end of the tubular portion, which hub portion is capable of attaching to a means for creating a vacuum in the tubular portion lumen. This new cannula, of the present invention, could best be described as having a tubular portion that is semi-flexible.

The means for creating a vacuum for use with the body tissue aspiration cannula of the invention can be either a manual vacuum syringe, plunger type, common for manual vacuum abortion, or an electrical mechanical vacuum pump as is typical in large hospital settings.

### BRIEF DESCRIPTION OF THE DRAWINGS

FIG. 1 is a side elevation view of an embodiment of the present invention.

FIG. 2 is an enlarged, fragmentary or cut away, side elevation view of the cannula showing the lumen and the hub attachment.

FIG. 3 is a partial perspective view of the hub portion showing a second embodiment of a hub portion of the cannula.

FIG. 4 is a perspective view of the winged hub portion of the invention.

### DETAILED DESCRIPTION OF THE INVENTION

The body tissue aspiration cannula of the present invention consists of a tubular portion made of a polymer material and a hub portion for attaching the tubular portion to a vacuum source. The polymer for the tubular portion needs to be a surgical grade polymer biocompatable for use in humans. Primarily, this means that the polymer should be biocompatible, for example, as demonstrated by passing or qualifying to pass the criteria of FDA 21 CFR 177.1520 or USP Class VI. The flexural modulus of the polymer selected for the tubular portion is also extremely important in balancing the use and flexibility of the tubular portion between the often too hard and the soft flexible tubes. Finally, the selected polymer for both the tubular portion and hub portion should be readily steam sterilizeable. That is, capable of repeated steam sterilization without substantial degradation of the polymer comprising either the tubular or hub portion of the cannula.

The flexural modulus of the polymer used in the tubular portion and hub portion of the present invention is measured by standard ASTM test D790 at 1% secant, 0.05 in/min, using Method A and measured in parts per square inch.

In one embodiment of the present invention, the flexural modulus of the tubular portion is between about 100,000 psi and 225,000 psi. In another embodiment, it is between 110,000 psi and 200,000 psi. In yet another embodiment, it is between 120,000 psi and 180,000 psi and in still another embodiment; it is between 130,000 psi and 150,000 psi.

Other properties of the selected tubular portion polymer material include tensile strength, which in one embodiment is in a range from about 2,000-8,000 psi as measured by ASTM D638 and can be chosen based on other parameters as desired. It is desirable that the polymer material chosen be easy to process and be able to be used in the standard processing methods for cannula tube production, such as extrusion, so processing (melt) temperatures should be in a range of about 400-600° F. The polymer material should be amendable by addition of whistle cuts, tip end sealing, and ethylene oxide sterilization (ETO) as well as the repeated steam sterilization of the polymer material as described above. The polymer should also have a Rockwell hardness (ASTM788) of about 60-75.

The tubular portion of the body tissue aspiration cannula is about 18-24 cm in length and will have a diameter ranging from about 4 mm to 12 mm in 1 mm increments or as desired for the particular use and situation. The distal end of the cannula tubing is sealed using a standard heat-sealing technique for surgical cannulae or other suitable means. In one embodiment, the selected polymer is clear or at least translucent in order for the user to be able to view the contents entering the tubular portions of the cannula.

The distal end of the tubing also will have one or more aspiration apertures for aspirating body tissue into the lumen of the tubular portion. These apertures are normally whistle cut, the width of the apertures being less than the width of the lumen to prevent large pieces of tissue from becoming lodged in the lumen of the tubular portion. The depth of the whistle cuts is generally from about half or less of the diameter of the tubing. The length of the whistle cut runs from about 150% to about 200% of the diameter of the cannula tubing. The whistle cut normally begins from about 3 to 5 mm from the sealed tip of the tubular portion.

If a second whistle cut is used, which is common in a tubular portion less than 9 mm in diameter, then such second whistle cut should start roughly where the most proximal end of the first whistle cut ends. The distal edges of each whistle cut are normally angled about 5% or so proximal to a line 45 degrees off the center axis of the tube itself. This allows the whistle cut to have an edge which when pulled across body tissue is capable of scraping body tissue from a selected area.

In a cannula of 9 mm in diameter and greater, normally only a single aperture is used. Whistle cuts are not as desirable in these larger sizes of cannulae due to the volume and size of aspirate encountered and so a scoop cut is employed. These cuts are usually of a depth half of the diameter of the cannula tubing and a width about 200-300% of the diameter of the cannula tubing.

While whistle and scoop cuts for aspiration apertures are relatively common on aspiration cannulae, polyethylene tubular portions may experience bending, kinking, or twisting of the tubing portion around the cuts due to the weakness introduced in the tube by such cuts. The tubular portion of the invention resists such bending, kinking, or twisting when compared to the tubular portion made of polyethylene when these same cuts are incorporated.

The thickness of the wall of the tubular portion will vary with the diameter of the tube but in general, the wall thickness will be about 0.25 mm to about 1.5 mm depending on the exact polymer used within the scope of the invention. The selection of the thickness of the tubular portion is within the skill in the art. The polymer meeting the above criteria can be a polypropylene homo or copolymer or any other polymer or polymer combinations meeting the criteria discussed above. Comparatively, the tubular portion of the invention exhibits improved performance when compared to known polyethylene tubing of the same wall thickness and outer diameter.

The proximal end of the tubular portion is attached to a cannula hub portion. The hub portion is essentially hollow and cylindrical with a lumen therethrough. The distal end of the lumen is fitted to the proximal end of the tubular portion and passes through to a hub portion proximal end for mounting to either a manual (vacuum syringe) or a powered source of vacuum in order to create a vacuum in the lumen of the tubular portion of the body tissue aspiration cannula. The hub portion can be used as needed as a grip surface when inserting or disconnecting the tubular portion to the vacuum source. The novel winged portion of one embodiment of the hub portion is useful in aiding such grip by helping prevent rotation of the hub. In order to maintain a vacuum within the lumen of the tubular portion, the distal end of the hub portion needs to be attached to the proximal end of the tubular portion such that there is a seal preventing leakage of any vacuum at the attachment point of the hub lumen to the lumen of the tubular portion. This attachment may be accomplished by any reasonable means in the art such as attaching the hub portion to the tubular portion by injection molding the hub portion directly on the tubular portion proximal end. Since it is anticipated that the cannula may be steam sterilized, like the tubing portion, the hub portion needs to be made of a material, such as a surgical grade polymer, that is both biocompatible and steam sterilizable. It is desirable that any polymer selected is relatively stiff and has thicker sidewalls than the tubular portion. A polymer such as the polyproplylene copolymer known as Profax SR549M (Basel Polyolefin Company) would be an example. This polymer has a flexural modulus of 150,000 psi. Hubs are approximately 20 to 50 mm in length; the stiffness necessary for the selected polymer of the hub can be selected based on increasing the wall thickness of the hub portion or by varying the modulus selected therefore.

In one embodiment of the hub portion of the cannula there is, in addition to the body portion, a pair of grip wings consisting of opposing planar attachments mounted toward the distal end of the hub and extending toward the distal end of the hub between about 10% and 50% the length of the hub. The width of the wings is as desired for comfortable grip by the fingers but in general between about 4 mm and about 20 mm. This hub has the advantage of easier grip due to the increase surface area as well as preventing slippage when attempting to disengage the cannula from the vacuum source.

In another embodiment of the larger cannulae, the hub portion is the proximal end of the tubular portion. That is, the proximal end of the tubular portion directly attaches to the vacuum source. In this embodiment, optional grip wings would attach directly to the tubular portion. This would be the case where the tubular portion is the same diameter as the mounting on the vacuum source.

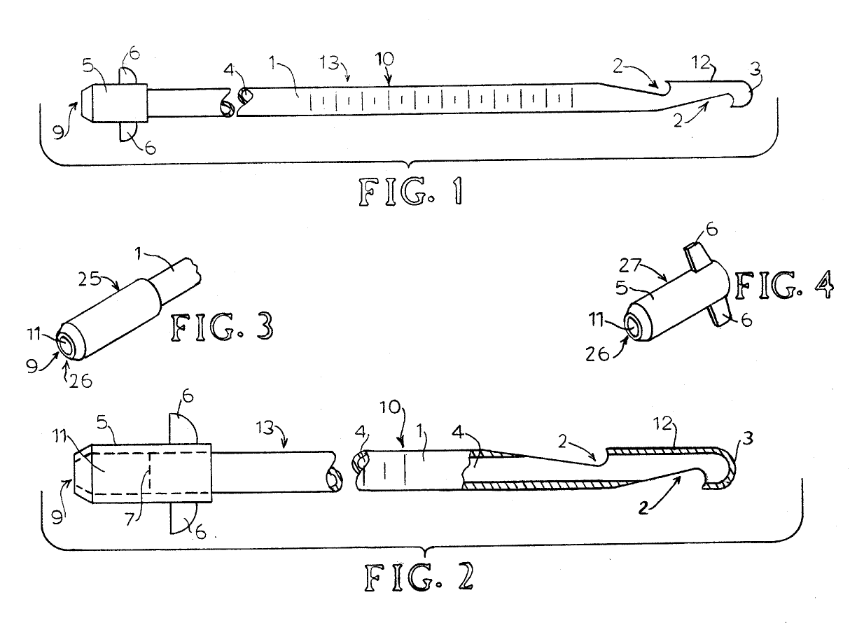

Referring now to FIGS. 1 through 4, embodiments of the present invention are shown. In FIG. 1, a side elevation view of an embodiment of the invention is shown. The body tissue aspiration cannula 13 consists of a tubular portion 1, constructed of a polymeric material in which two opposing whistle-cut aspiration apertures 2 are shown toward its distal end 3. Each of the aspiration apertures 2 are prepared in a whistle cut design. The distal end 3 of tubular portion 1 is sealed in order that vacuum may be created in lumen 4 of the tubular portion 1. The proximal end 7, as seen in FIG. 2, of tubular portion 1 is attached to a mounting/gripping hub portion 5 for attachment to a selected vacuum source (not shown). This embodiment shows hub portion 5 with a pair of opposing grip wings 6. Further, depth markings 10 on tubular portion 1 are used to indicate the depth tubular portion 1 is inserted into the uterine cavity during use.

In FIG. 2, an enlarged, cut-away view of the body tissue aspiration cannula 13 in FIG. 1 is shown. Lumen 4 is shown more extensively than in FIG. 1 and a cut-away view depicts the attachment of tubular portion 1 and hub portion 5. Also shown, are whistle-cut aspiration apertures 2 cut into cannula tubing sidewall 12 and lumen 4. Sealed distal end 3 is also shown in cut away view. The proximal end 7 is attached to a mounting/gripping cannula hub portion 5 with grip wings 6. In the cut-away, it is clear proximal end 7 is inside lumen 11 of hub portion 5. The proximal end 9 of hub portion 5 is open and is the means for attaching to a manual or mechanical vacuum source. Tubular portion 1 has a plurality of depth markings 10 for observation and measurement by the practitioner using the body tissue aspiration cannula 13 during insertion as an indication of the depth of the penetration of the cannula 13 into the uterus.

FIG. 3 is a partial perspective view of another embodiment of the hub portion 25 attached to a proximal end of a tubular portion 1. In this view, tubular portion 1 is attached to hub portion 25. In this embodiment, there are no grip wings as in FIGS. 1 and 2. Also shown in this perspective is the lumen 11 of the hub portion 25 which allows for vacuum creation when the cannula is attached to the vacuum source. Further shown is the attachment means 26.

FIG. 4 is a perspective view of a winged hub portion of the invention. In this perspective hub portion 5 has a body portion 27 having a lumen 11 and an attachment means 26 for attaching to a vacuum device which allows for vacuum creation when the cannula is attached to a vacuum source. Of particular distinction are grip wings 6 which uniquely aid in gripping the hub portion 5 when attaching or disengaging the cannula to the vacuum source and prevent its rotation while gripping especially when the hub is wet.

### EXAMPLE

Polypropylene copolymer Profax® SR256M (Basel Polyolefin Company) is selected for the tubular portion of a body tissue aspiration cannula. SR256M has a flexural modulus of 140,000 psi using ASTM D790 at 1% secant, 0.05 in/min and using Method A. Further, it has a Rockwell hardness of 69, a processing (melt) temperature of 475-525° F., a melt flow rate of about 2.0 g/10 min and a tensile strength at yield of 4000 psi. The polymer is also steam and chemically sterilzable. The tube is approximately 9 cm long, 7 mm in diameter and has a wall thickness of about 75 mm. A hub portion is injection molded onto the tubular portion suitable for attaching to a manual vacuum (aspiration) device such as a vacuum syringe. The hub has opposing grip wings as in FIG. 4 and is made from Profax SR549M polypropylene copolymer. The distal end is sealed and two aspiration apertures cut, whistle style in the distal end of the tubular portion, to form a cannula as embodied in FIGS. 1 and 2. The body tissue cannula of the invention was vertical load tested at the distal end, against a cannula having a tubular portion made of polyethylene for kink resistance. The cannula of the invention was able to withstand approximately 13.2 pounds force as compared with the known polyethylene cannula which supported 3.3 pounds of pressure.

The Example and disclosure as well as the other information herein are not intended to be limiting. Selection of polymers, production means, use and the like are well within the scope of the art and those so skilled in the art would be able to so choose in light of the following claims and the teachings in the specification.

What is claimed is:

1. A body tissue aspiration cannula comprising a tubular portion having a proximal end, a distal end and a lumen extending therebetween, said distal end being sealed, the tubular portion comprising a biocompatible, steam sterilizable polymer having a flexural modulus of from about 100,000 psi to about 225,000 psi, said tubular portion having one or more body tissue aspiration apertures disposed near said distal end and a hub portion comprising a biocompatible, steam sterilizable polymer, is secured to said tubular portion proximal end and wherein said hub portion is capable of attaching to a means for creating a vacuum in said tubular portion lumen.

2. A body tissue aspiration cannula according to claim 1 wherein the tubular portion polymer is a polypropylene mono or copolymer.

3. A body tissue aspiration cannula according to claim 1, having an outer diameter of from about 4 mm to about 12 mm.

4. A body tissue aspiration cannula according to claim 1, which is further attached by said hub to a manual vacuum syringe or an electric vacuum source.

5. A body tissue aspiration cannula according to claim 1, wherein said aspiration aperture is a whistle cut, said cut having a width no larger than half the diameter of said lumen.

6. A body tissue aspiration cannula according to claim 2 wherein said polypropylene has a Rockwell hardness of from about 64 to about 74.

7. A body tissue aspiration cannula according to claim 6, wherein said Rockwell hardness is about 69.

8. A body tissue aspiration cannula according to claim 1, wherein said flexural modulus is about 140,000 psi.

9. A body tissue aspiration cannula according to claim 1, wherein there are two apertures disposed near said distal end.

10. A body tissue aspiration cannula according to claim 2 wherein said polypropylene has a melt flow rate of about 1.0-10.0 g/10 min.

11. A body tissue aspiration cannula according to claim 10, wherein said polypropylene has a melt flow rate of about 2.0 g/10 min.

12. A body tissue aspiration cannula according to claim 1, wherein said hub portion further comprises a pair of opposing grip wings.

13. A body tissue aspiration cannula according to claim 2, wherein said polypropylene has a flexural modulus of from about 110,000 to about 200,000 psi.

14. A body tissue aspiration cannula according to claim 2, wherein said polypropylene has a flexural modulus of from about 120,000 to about 180,000 psi.

15. A body tissue aspiration cannula according to claim 2, wherein said polypropylene has a flexural modulus of from about 130,000 to about 150,000 psi.

16. An aspiration cannula comprising a semi-flexible polymer tubular portion having a proximal end and a distal end and a lumen extending therebetween, said distal end being sealed, said tubular portion comprising one or more apertures positioned near said distal end and a hub portion secured to said proximal end of said tubular portion capable of attaching to a means for creating a vacuum in said lumen.

17. An abortion tissue aspiration cannula comprising, a tubular portion having a proximal end, distal end and a lumen extending therebetween, said distal end being sealed, said tubular portion comprising a biocompatible, steam sterilizeable polymer having a flexural modulus of about 140,000 psi, said tubular portion having one or more aspiration apertures disposed near said distal end and wherein a hub portion is secured to said proximal end of said tubular portion proximal end, said hub portion capable of attaching to a means for creating a vacuum in said lumen.

18. An abortion tissue aspiration cannula according to claim 17, wherein the hub portion further comprises a pair of opposing grip wings.

19. An abortion tissue aspiration cannula according to claim 17 wherein there are two aspiration apertures disposed near said distal end.

20. An abortion tissue aspiration cannula according to claim 17 wherein there are a series of graduation markings on said tubular portion.

21. A cannula hub for attaching a cannula tubular portion to a vacuum source comprising a body portion having a lumen extending therethrough, a proximal end of said lumen designed to attach to a vacuum source and a distal end of said lumen designed to attach to a cannula tubular portion, said cannula hub further comprising a pair of opposing grip wings disposed at a distal end of said cannula hub.
